(19) United States
(12) Patent Application Publication
(10) Pub. No.: US 2023/0240740 A1
(43) Pub. Date: Aug. 3, 2023

(54) THERMAL ELEMENTS FOR SURGICAL INSTRUMENTS AND SURGICAL INSTRUMENTS INCORPORATING THE SAME

(71) Applicant: Covidien LP, Mansfield, MA (US)

(72) Inventors: William E. Robinson, Boulder, CO (US); James D. Allen, IV, Broomfield, CO (US)

(21) Appl. No.: 18/092,659

(22) Filed: Jan. 3, 2023

Related U.S. Application Data
(60) Provisional application No. 63/305,493, filed on Feb. 1, 2022.

(51) Int. Cl. A61B 18/08

(52) U.S. Cl. CPC A61B 18/085; A61B 2018/00714

(57) ABSTRACT

A thermal element configured for thermally treating tissue includes a substrate, an insulating layer disposed on the substrate, and a resistive trace circuit disposed on the insulating layer and including first and second ends adapted to connect to a source of energy for energizing the resistive trace circuit, thereby heating the thermal element. The resistive trace circuit: includes at least first and second sections defining different configurations and disposed between the first and second ends; is configured to maintain a temperature variation of no greater than about 25° C. along at least a majority of a length of the thermal element; and/or is configured to define a temperature gradient profile that varies at least about 50° C. along a length of the thermal element at a target operating temperature thereof.

### CROSS-REFERENCE TO RELATED APPLICATIONS

This application claims the benefit of, and priority to, U.S. Provisional Patent Application No. 63/305,493, filed on Feb. 1, 2022, the entire contents of which are hereby incorporated herein by reference.

### FIELD

The present disclosure relates to surgical instruments and, more particularly, to thermal elements for surgical instruments and surgical instruments incorporating the same.

### BACKGROUND

A surgical forceps is a pliers-like instrument that relies on mechanical action between its jaw members to grasp, clamp, and constrict tissue. Electrosurgical forceps utilize both mechanical clamping action and energy to heat tissue to treat, e.g., coagulate, cauterize, or seal, tissue. Typically, once tissue is treated, the surgeon has to accurately sever the treated tissue. Accordingly, many electrosurgical forceps are designed to incorporate a knife that is advanced between the jaw members to cut the treated tissue. As an alternative to a mechanical knife, an energy-based tissue cutting element may be provided to cut the treated tissue using energy, e.g., thermal, electrosurgical, ultrasonic, light, or other suitable energy.

Energy-based elements are also utilized in various other surgical instruments and/or to otherwise facilitate treating tissue, e.g., coagulating tissue, sealing tissue, cutting tissue, etc., using energy, e.g., thermal, electrosurgical, ultrasonic, light, or other suitable energy.

### SUMMARY

As used herein, the term “distal” refers to the portion that is being described which is farther from an operator (whether a human surgeon or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, design variations, and/or other variations, up to and including plus or minus 10 percent. Further, to the extent consistent, any or all of the aspects detailed herein may be used in conjunction with any or all of the other aspects detailed herein.

Provided in accordance with aspects of the present disclosure is a thermal element configured for thermally treating tissue and including a substrate, an insulating layer disposed on the substrate, and a resistive trace circuit disposed on the insulating layer. The resistive trace circuit includes first and second ends adapted to connect to a source of energy for energizing the resistive trace circuit, thereby heating the thermal element (e.g., via resistive heating (Joule heating)). The resistive trace circuit further includes at least first and second sections between the first and second ends. The first and second sections define different configurations.

In an aspect of the present disclosure, the first section of the resistive trace circuit defines a substantially linear configuration, and the second section of the resistive trace circuit defines a tortuous configuration. The tortuous configuration may define a repeating pattern having a uniform frequency or a varied frequency.

In another aspect of the present disclosure, the first and second ends of the resistive trace circuit are disposed at a first end portion of the substrate. In such aspects, the resistive trace circuit extends from the first end thereof at the first end portion of the substrate to a return portion (which may be part of the resistive trace circuit) at a second end portion of the substrate and back from the return portion at the second end portion of the substrate to the second end of the resistive trace circuit at the first end portion of the substrate.

In yet another aspect of the present disclosure, the first section of the resistive trace circuit is disposed between the first end of the resistive trace circuit and the return portion of the resistive trace circuit, and the second section of the resistive trace circuit is disposed between the return portion of the resistive trace circuit and the second end of the resistive trace circuit.

In still another aspect of the present disclosure, only one of the first or second sections of the resistive trace circuit includes at least one temperature control feature. The at least one temperature control feature may include electrically-conductive material disposed on the resistive trace circuit and/or a variation in cross-sectional thickness of the resistive trace circuit.

In still yet another aspect of the present disclosure, the resistive trace circuit further includes a third section between the first and second ends. In such aspects, the third section defines a different configuration from both the first section and the second section.

In another aspect of the present disclosure, one of the first, second, or third sections includes a substantially linear configuration; another of the first, second, or third sections includes a tortuous configuration; and still another of the first, second, or third sections includes at least one temperature control feature.

Another thermal element provided in accordance with aspects of the present disclosure and configured for thermally treating tissue includes a substrate, an insulating layer disposed on the substrate, and a resistive trace circuit disposed on the insulating layer. The resistive trace circuit includes first and second ends (in aspects, electrically-conductive, non-resistive ends, or conductive (non-resistive) traces connected to the ends) adapted to connect to a source of energy for energizing the resistive trace circuit, thereby heating the thermal element. The resistive trace circuit is configured to maintain a temperature variation of no greater than about 25° C. along at least a majority of a length of the thermal element at a target operating temperature thereof.

In an aspect of the present disclosure, a first section of the resistive trace circuit defines a substantially linear configuration, and a second section of the resistive trace circuit defines a tortuous configuration.

In another aspect of the present disclosure, the first and second ends of the resistive trace circuit are disposed at a first end portion of the substrate, and the resistive trace circuit extends from the first end thereof at the first end portion of the substrate to a return portion at a second end portion of the substrate and back from the return portion at the second end portion of the substrate to the second end of the resistive trace circuit at the first end portion of the substrate.

In yet another aspect of the present disclosure, a first section of the resistive trace circuit disposed between the first end of the resistive trace circuit and the return portion of the resistive trace circuit defines a different configuration from a second section of the resistive trace circuit disposed between the return portion of the resistive trace circuit and the second end of the resistive trace circuit.

In still another aspect of the present disclosure, the resistive trace circuit includes at least one temperature control feature.

Another thermal element provided in accordance with aspects of the present disclosure and configured for thermally treating tissue includes a substrate, an insulating layer disposed on the substrate, and a resistive trace circuit disposed on the insulating layer. The resistive trace circuit includes first and second ends adapted to connect to a source of energy for energizing the resistive trace circuit, thereby heating the thermal element. The resistive trace circuit is configured to define a temperature gradient profile that varies at least about 50° C. along a length of the thermal element at a target operating temperature thereof.

In an aspect of the present disclosure, a first section of the resistive trace circuit defines a substantially linear configuration, and a second section of the resistive trace circuit defines a tortuous configuration.

In another aspect of the present disclosure, the first and second ends of the resistive trace circuit are disposed at a first end portion of the substrate. In such aspects, the resistive trace circuit extends from the first end thereof at the first end portion of the substrate to a return portion at a second end portion of the substrate and back from the return portion at the second end portion of the substrate to the second end of the resistive trace circuit at the first end portion of the substrate.

In still another aspect of the present disclosure, the temperature gradient profile includes at least one peak disposed between the first and second ends of the conducive circuit trace at the target operating temperature.

In yet another aspect of the present disclosure, the temperature gradient profile includes at least two peaks disposed between the first and second ends of the conducive circuit trace at the target operating temperature.

### BRIEF DESCRIPTION OF DRAWINGS

The above and other aspects and features of the present disclosure will become more apparent in view of the following detailed description when taken in conjunction with the accompanying drawings wherein like reference numerals identify similar or identical elements.

FIG. 1 is a perspective view of a shaft-based electrosurgical forceps provided in accordance with the present disclosure shown connected to an electrosurgical generator;

FIG. 2 is a perspective view of a hemostat-style electrosurgical forceps provided in accordance with the present disclosure;

FIG. 3 is a schematic illustration of a robotic surgical instrument provided in accordance with the present disclosure;

FIG. 4 is a perspective view of an end effector assembly of the forceps of FIG. 1 including first and second jaw members;

FIG. 5 is a perspective view of the thermal element of the second jaw member of the end effector assembly of FIG. 4;

FIGS. 6-10 are side views of portions of various other thermal elements provided in accordance with aspects of the present disclosure; and

FIG. 11 is a graph illustrating temperature gradients of the thermal elements of FIGS. 5-10 along the lengths of the respective thermal elements.

### DETAILED DESCRIPTION

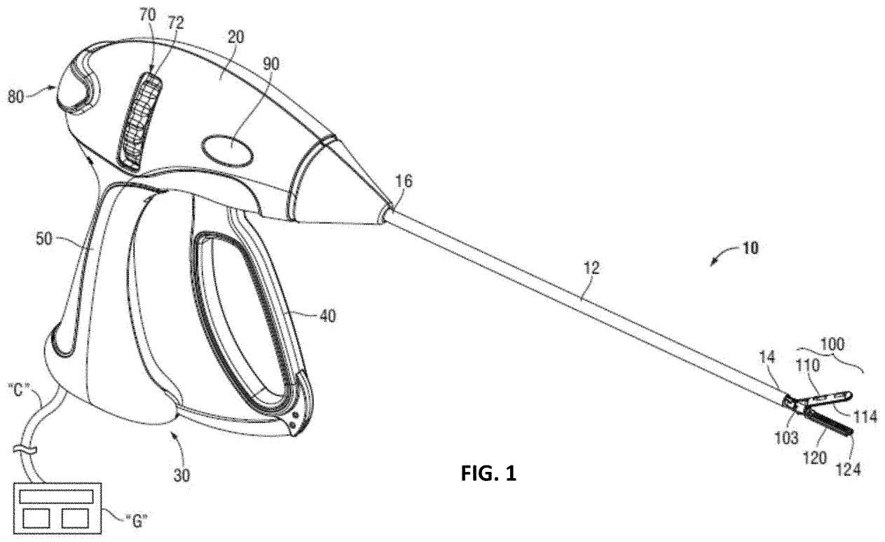

Referring to FIG. 1, a shaft-based electrosurgical forceps provided in accordance with the present disclosure is shown generally identified by reference numeral 10. Aspects and features of forceps 10 not germane to the understanding of the present disclosure are omitted to avoid obscuring the aspects and features of the present disclosure in unnecessary detail.

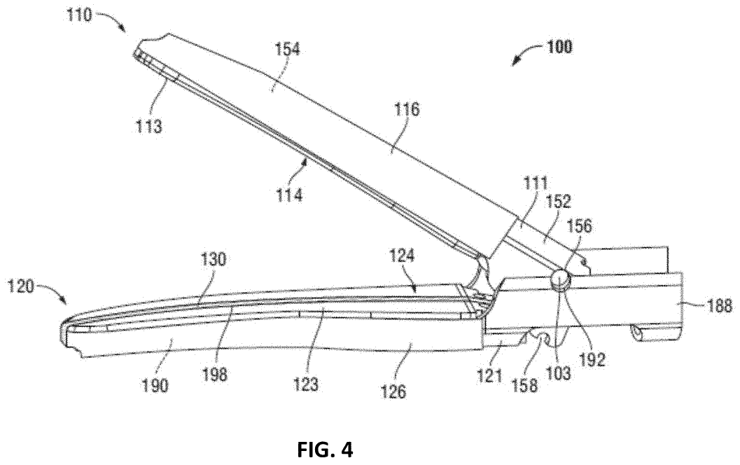

Forceps 10 includes a housing 20, a handle assembly 30, a rotating assembly 70, a first activation switch 80, a second activation switch 90, and an end effector assembly 100. Forceps 10 further includes a shaft 12 having a distal end portion 14 configured to (directly or indirectly) engage end effector assembly 100 and a proximal end portion 16 that (directly or indirectly) engages housing 20. Forceps 10 also includes cable “C” that connects forceps 10 to an energy source, e.g., an electrosurgical generator “G.” Cable “C” includes a wire (or wires) (not shown) extending therethrough that has sufficient length to extend through shaft 12 in order to connect to one or both tissue treating surfaces 114, 124 of jaw members 110, 120, respectively, of end effector assembly 100 to provide energy thereto and/or a wire (or wires) (not shown) extending through cable “C” that has sufficient length to extend through shaft 12 in order to connect to thermal element 130 (FIG. 4). Alternatively, forceps 10 may be configured as a cordless device such as, for example, including an on-board power source, e.g., a DC battery, and an on-board electrosurgical generator (not shown) powered by the on-board power source. The power source may provide energy to thermal element 130 (FIG. 4) and the electrosurgical generator may provide electrosurgical energy to tissue treating surfaces 114, 124, although the electrosurgical generator “G” may also provide the energy to thermal element 130 (FIG. 4). In other configurations, forceps 10 includes an on-board power source for providing energy to thermal element 130 (FIG. 4) and connects to electrosurgical generator “G” via cable “C” for providing electrosurgical energy to tissue treating surfaces 114, 124.

First activation switch 80 is coupled to tissue treating surfaces 114, 124 and the electrosurgical generator “G” for enabling the selective activation of the supply of energy to jaw members 110, 120 for treating, e.g., cauterizing, coagulating/desiccating, and/or sealing, tissue. Second activation switch 90 is coupled to thermal element 130 of jaw member 120 (see FIG. 4) and the electrosurgical generator “G” for enabling the selective activation of the supply of energy to thermal element 130 (FIG. 4) for thermally treating, e.g., cutting, tissue.

Handle assembly 30 of forceps 10 includes a fixed handle 50 and a movable handle 40. Fixed handle 50 is integrally associated with housing 20 and handle 40 is movable relative to fixed handle 50. Movable handle 40 of handle assembly 30 is operably coupled to a drive assembly (not shown) that, together, mechanically cooperate to impart movement of one or both of jaw members 110, 120 of end effector assembly 100 about a pivot 103 between a spaced apart position and an approximated position to grasp tissue between tissue treating surfaces 114, 124 of jaw members 110, 120. As shown in FIG. 1, movable handle 40 is initially spaced apart from fixed handle 50 and, correspondingly, jaw members 110, 120 of end effector assembly 100 are disposed in the spaced apart position. Movable handle 40 is depressible from this initial position towards fixed handle 50 to a depressed position corresponding to the approximated position of jaw members 110, 120. Rotating assembly 70 includes a rotation wheel 72 that is selectively rotatable in either direction to correspondingly rotate shaft 12 and end effector assembly 100 relative to housing 20.

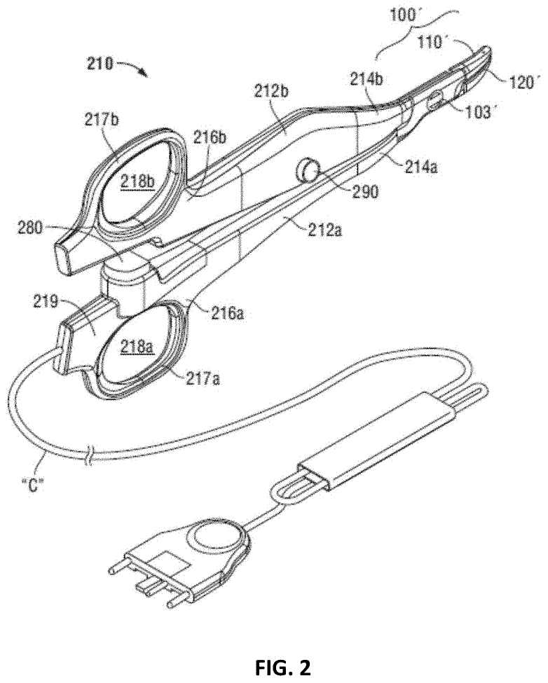

Referring to FIG. 2, a hemostat-style electrosurgical forceps provided in accordance with the present disclosure is shown generally identified by reference numeral 210. Aspects and features of forceps 210 not germane to the understanding of the present disclosure are omitted to avoid obscuring the aspects and features of the present disclosure in unnecessary detail.

Forceps 210 includes two elongated shaft members 212a, 212b, each having a proximal end portion 216a, 216b, and a distal end portion 214a, 214b, respectively. Forceps 210 is configured for use with an end effector assembly 100′ similar to and including any of the features of end effector assembly 100 (FIGS. 1 and 4). More specifically, end effector assembly 100′ includes first and second jaw members 110′, 120′ attached to respective distal end portions 214a, 214b of shaft members 212a, 212b. Jaw members 110′, 120′ are pivotably connected about a pivot 103′. Each shaft member 212a, 212b includes a handle 217a, 217b disposed at the proximal end portion 216a, 216b thereof. Each handle 217a, 217b defines a finger hole 218a, 218b therethrough for receiving a finger of the user. Finger holes 218a, 218b facilitate movement of the shaft members 212a, 212b relative to one another to, in turn, pivot jaw members 110′, 120′ from the spaced apart position, wherein jaw members 110′, 120′ are disposed in spaced relation relative to one another, to the approximated position, wherein jaw members 110′, 120′ cooperate to grasp tissue therebetween.

One of the shaft members 212a, 212b of forceps 210, e.g., shaft member 212a, includes a proximal shaft connector 219 configured to connect forceps 210 to a source of energy, e.g., electrosurgical generator “G” (FIG. 1). Proximal shaft connector 219 secures a cable “C” to forceps 210 such that the user may selectively supply energy to jaw members 110′, 120′ for treating tissue. More specifically, a first activation switch 280 is provided on one of the shaft members, e.g., shaft member 212a, for supplying energy to jaw members 110′, 120′ to treat tissue upon sufficient approximation of shaft members 212a, 212b, e.g., upon activation of first activation switch 280 via the other shaft member 212b. A second activation switch 290 disposed on either or both of shaft members 212a, 212b is coupled to the thermal element (not shown, similar to thermal element 130 of jaw member 120 (FIG. 4)) of one of the jaw members 110′, 120′ of end effector assembly 100′ and to the electrosurgical generator “G” (or other suitable power source) for enabling the selective activation of the supply of energy to the thermal element for thermally treating, e.g., cutting, tissue. Alternatively, second activation switch 290 may be omitted and the electrosurgical generator “G” (FIG. 1) may be configured to automatically power the thermal cutter when appropriate. In other aspects, second activation switch 290 is provided and electrosurgical generator “G” (FIG. 1) provides control based on jaw position, handle position, and the like to enable or disable activation.

Jaw members 110′, 120′ define a curved configuration wherein each jaw member is similarly curved laterally off of a longitudinal axis of end effector assembly 100′. However, other suitable curved configurations including curvature towards one of the jaw members 110′, 120′ (and thus away from the other), multiple curves with the same plane, and/or multiple curves within different planes are also contemplated. Jaw members 110, 120 of end effector assembly 100 (FIG. 1) may likewise be curved according to any of the configurations noted above or in any other suitable manner.

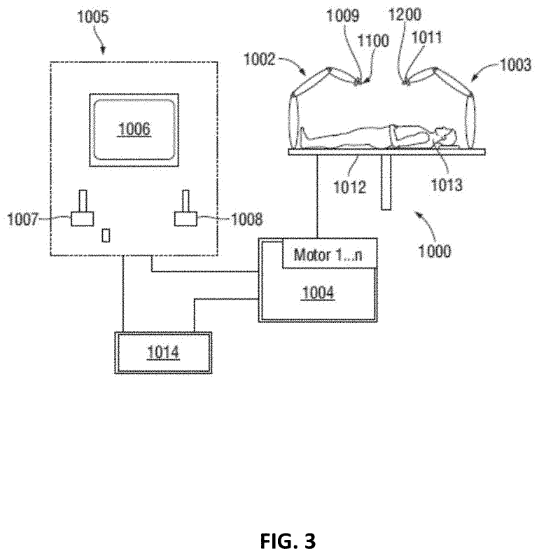

Referring to FIG. 3, a robotic surgical instrument provided in accordance with the present disclosure is shown generally identified by reference numeral 1000. Aspects and features of robotic surgical instrument 1000 not germane to the understanding of the present disclosure are omitted to avoid obscuring the aspects and features of the present disclosure in unnecessary detail.

Robotic surgical instrument 1000 includes a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a surgeon may be able to telemanipulate robot arms 1002, 1003 in an operating mode. Robotic surgical instrument 1000 may be configured for use on a patient 1013 lying on a patient table 1012 to be treated in a minimally invasive manner. Robotic surgical instrument 1000 may further include or be capable of accessing a database 1014, in particular coupled to control device 1004, in which are stored, for example, pre-operative data from patient 1013 and/or anatomical atlases.

Each of the robot arms 1002, 1003 may include a plurality of members, which are connected through joints, and an attaching device 1009, 1011, to which may be attached, for example, an end effector assembly 1100, 1200, respectively. End effector assembly 1100 is similar to and may include any of the features of end effector assembly 100 (FIGS. 1 and 4), although other suitable end effector assemblies for coupling to attaching device 1009 are also contemplated. End effector assembly 1200 may be any end effector assembly, e.g., an endoscopic camera, other surgical tool, etc. Robot arms 1002, 1003 and end effector assemblies 1100, 1200 may be driven by electric drives, e.g., motors, that are connected to control device 1004. Control device 1004 (e.g., a computer) may be configured to activate the motors, in particular by means of a computer program, in such a way that robot arms 1002, 1003, their attaching devices 1009, 1011, and end effector assemblies 1100, 1200 execute a desired movement and/or function according to a corresponding input from manual input devices 1007, 1008, respectively. Control device 1004 may also be configured in such a way that it regulates the movement of robot arms 1002, 1003 and/or of the motors.

Turning to FIG. 4, end effector assembly 100, as noted above, includes first and second jaw members 110, 120. Either or both jaw members 110, 120 may include a structural frame 111, 121, an insulative spacer (not shown), a tissue treating plate 113, 123 defining the respective tissue treating surface 114, 124 thereof, and, in aspects, an outer insulative jacket 116, 126. Tissue treating plates 113, 123 may be pre-formed and engaged with the insulative spacers and/or other portion(s) of jaw members 110, 120 via, for example, overmolding, adhesion, mechanical engagement, etc., or may be deposited onto the insulative spacers, e.g., via sputtering or other suitable deposition technique. As utilized herein, the term “insulative” may refer to electrical and/or thermal insulation except as specified otherwise.

Jaw member 110, as noted above, includes a structural frame 111, an insulative spacer (not shown, in other aspects, the insulative spacer is omitted or integrated into structural frame 111), a tissue treating plate 113 defining tissue treating surface 114, and, in aspects, an outer insulative jacket 116. Structural frame 111 may be formed from stainless steel or other suitable material configured to provide structural support to jaw member 110. Structural frame 111 includes a proximal flange portion 152 about which jaw member 110 is pivotably coupled to jaw member 120 via pivot 103 and a distal body portion 154 that supports the other components of j aw member 110, e.g., the insulative spacer, tissue treating plate 113, and outer insulative jacket 116 (where provided). In shaft-based or robotic configurations, proximal flange portion 152 enables operable coupling of jaw member 110 to the drive assembly (not shown) to enable pivoting of jaw member 110 relative to jaw member 120 in response to actuation of the drive assembly. More specifically, proximal flange portion 152 may define an aperture 156 for receipt of pivot 103 and at least one catch 158 for receipt of a drive pin of the drive assembly (not shown) such that translation of the drive pin, e.g., in response to actuation of movable handle 40 (FIG. 1) or a robotic drive, pivots jaw member 110 about pivot 103 and relative to jaw member 120 between the spaced apart position and the approximated position. However, other suitable drive arrangements are also contemplated, e.g., using cam pins and cam slots, a screw-drive mechanism, etc. In hemostat-style devices, proximal flange portion 152 is secured to one of the shaft members, e.g., shaft member 212a of forceps 210 (see FIG. 2). Proximal flange portion 152 may be bifurcated to define a pair of spaced apart proximal flange portion segments or may otherwise be configured.

Distal body portion 154 of structural frame 111 extends distally from proximal flange portion 152 to support the other components of jaw member 110. The insulative spacer of jaw member 110 is supported on distal body portion 154 of structural frame 111 and is formed from an electrically insulative material capable of withstanding high temperatures such as, for example, up to at least 400° C. or 600° C., although other configurations are also contemplated. The insulative spacer may be formed from ceramic or other suitable material, e.g., PTFE, PEEK, PEI, etc. Tissue treating plate 113 is supported or received on the insulative spacer and is electrically connected, e.g., via one or more electrical leads (not shown), to first activation switch 80 (FIG. 1) and electrosurgical generator “G” (FIG. 1) to enable selective energization of tissue treating plate 113, e.g., as one pole of a bipolar Radio Frequency (RF) electrosurgical circuit. However, other suitable energy modalities, e.g., thermal, ultrasonic, light, microwave, infrared, etc., are also contemplated. The insulative spacer serves to electrically isolate structural frame 111 and tissue treating plate 113 from one another.

Continuing with reference to FIG. 4, jaw member 120 includes a structural frame 121, an insulative spacer (not shown, in other aspects the insulative spacer is omitted or integrated into structural frame 121), a tissue treating plate 123 defining tissue treating surface 124, and, in aspects, an outer insulative jacket 126. Jaw member 120 further include thermal element 130. Structural frame 121 of j aw member 120 defines a proximal flange portion 188 and a distal body portion 190 extending distally from proximal flange portion 188. Proximal flange portion 188 may be bifurcated to define a pair of spaced apart proximal flange portion segments or may define any other suitable configuration. Proximal flange portion 188 of jaw member 120 and proximal flange portion 152 of jaw member 110 may define a nestled configuration, e.g., wherein one of the proximal flange portions 152, 188 is received within the other, an overlapping configuration, e.g., wherein proximal flange portions 152, 188 at least partially overlap one another, or an offset configuration, e.g., wherein proximal flange portions 152, 188 are positioned in side-by-side relation. Regardless of the particular arrangement of proximal flange portions 152, 188, proximal flange portion 188 further defines a cut out 192 configured for receipt of pivot 103, e.g., welded or otherwise secured therein, to pivotably couple jaw members 110, 120 with one another. As an alternative to cut out 192, a hole or other suitable feature may be provided to enable coupling of pivot 103 with proximal flange portion 188. Proximal flange portion 188 may be secured to shaft 12 (FIG. 1) in shaft-based configurations (or a corresponding shaft portion in robotic configurations); alternatively, a bilateral configuration may be provided whereby both jaw member 110 and jaw member 120 are pivotable relative to shaft 12 (FIG. 1). In hemostat-style configurations, proximal flange portion 188 may be secured to elongated shaft 212b (FIG. 2).

The insulative spacer of jaw member 120 is supported on distal body portion 190 of structural frame 121 and is formed from an insulative material capable of withstanding high temperatures such as, for example, up to at least 400° C. or 600° C., although other configurations are also contemplated. The insulative spacer may be formed from ceramic or other suitable material, e.g., PTFE, PEEK, PEI. Tissue treating plate 123 is supported or received on the insulative spacer. Tissue treating plate 123, in particular, defines a longitudinally extending slot 198 therethrough along at least a portion of the length thereof. Slot 198 may be transversely centered on tissue treating surface 124 or may be offset relative thereto and may be linear, curved, include angled sections, etc. similarly or differently from the configuration, e.g., curvature, of jaw member 120. Slot 198 exposes a portion of thermal element 130, which may be recessed relative to tissue treating surface 124, substantially co-planar with tissue treating surface 124, or protrude beyond tissue treating surface 124 towards jaw member 110. In aspects where thermal element 130 protrudes, thermal element 130 may contact an opposing portion of jaw member 110 to set a minimum gap distance, e.g., of from about 0.001 inches to about 0.010 inches, between tissue treating surfaces 114, 124 in the approximated position of jaw members 110, 120.

Tissue treating plate 123 is electrically connected, e.g., via one or more electrical leads (not shown), to first activation switch 80 (FIG. 1) and electrosurgical generator “G” (FIG. 1) to enable selective energization of tissue treating plate 123, e.g., as the other pole of the bipolar (RF) electrosurgical circuit including tissue treating plate 113. In this manner, in the approximated position of jaw members 110, 120 grasping tissue therebetween, bipolar RF electrosurgical energy may be conducted between tissue treating plates 113, 123 and through the grasped tissue to treat, e.g., seal, the grasped tissue. However, other suitable energy modalities, e.g., thermal, ultrasonic, light, microwave, infrared, etc., are also contemplated, as are other suitable tissue treatments, e.g., coagulation.

Thermal element 130 may be secured within and directly to the insulative spacer 122 of jaw member 120 in any suitable manner, e.g., adhesive, friction fitting, overmolding, mechanical engagement, etc., or may be indirectly secured relative to the insulative spacer (in contact with or spaced apart therefrom) via attachment to one or more other components of jaw member 120. Alternatively, the insulative spacer may be omitted and thermal element 130 secured within jaw member 120 (to one or more components thereof) in any other suitable manner. Other suitable configurations for supporting thermal element 130 within jaw member 120 are also contemplated. Thermal element 130 may protrude distally beyond the distal tip of the insulative spacer of jaw member 120 (thus defining the distal-most extent of jaw member 120), may be substantially flush therewith, or may be recessed relative thereto. In aspects where end effector assembly 100, or a portion thereof, is curved, thermal element 130 may similarly be curved.

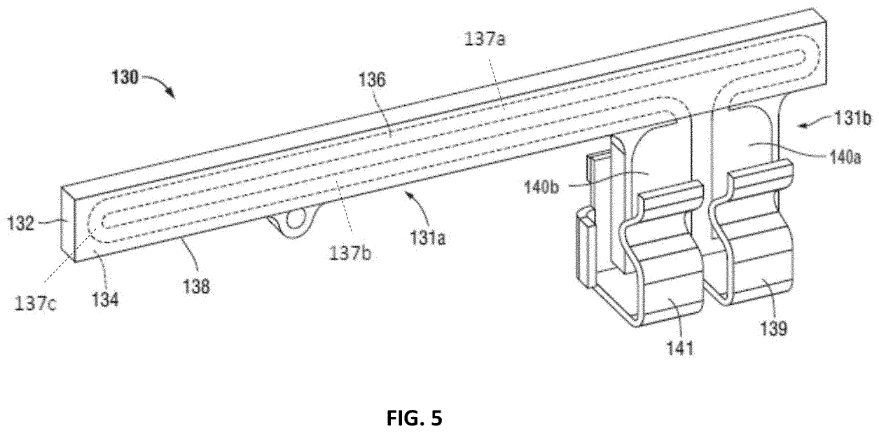

With additional reference to FIG. 5, thermal element 130 includes a body 131a and a proximal extension 131b. Thermal element 130 is formed from a base substrate 132 and includes an insulating layer 134 disposed on at least one side of base substrate 132, and a resistive trace circuit 136 disposed on insulating layer 134 on at least one side of base substrate 132. Resistive trace circuit 136 extends distally along body 131a of thermal element 130 and loops back proximally such that first and second ends 140a, 140b of resistive trace circuit 136 are disposed at proximal extension 131b of thermal element 130. First and second ends 140a and 140b may include a layer of electrically-conductive material to facilitate electrical connection to resistive trace circuit 136 for powering thermal element 130, e.g., via welding lead wires, mechanical connectors, etc. Ends 140a, 140b may additionally or alternatively include electrically-conductive lead traces to prevent heating of the proximal section of thermal element 130. First and second contact clips 139, 141 (or other suitable electrical connections such as welded lead wires) are coupled to proximal extension 131b of thermal element 130 in electrical communication with first and second ends 140a, 140b, respectively, of resistive trace circuit 136 for connecting lead wires (not shown) to thermal element 130 to enable application of an AC voltage thereto to heat thermal element 130, e.g., via resistive heating. More specifically, the lead wires electrically connect thermal element 130 to electrosurgical generator “G” (FIG. 1) (and, in aspects, also second activation switch 90 (FIG. 1) (if so provided) and/or first activation switch 80 (FIG. 1)) to enable selective activation of the supply of an AC voltage to thermal element 130 for heating thermal element 130 to heat and thereby thermally treat, e.g., cut, tissue. Thermal element 130 may be configured to cut previously (or concurrently) sealed tissue grasped between jaw members 110, 120, tissue extending across jaw member 120, tissue adjacent the distal end of jaw member 120, etc. In addition to or as an alternative to cutting, thermal element 130 may be configured for other tissue treatment, e.g., coagulation, sealing, dissection, etc.

Base substrate 132 may be formed from any suitable material such as, for example, ceramic, stainless steel, aluminum, aluminum alloys, titanium, titanium alloys, other suitable materials, combinations thereof, etc. Base substrate 132 may be formed via laser cutting, machining, casting, forging, fine-blanking, or any other suitable method. Base substrate 132 may define a thickness of, in aspects, from about 0.003 in to about 0.030 in; in other aspects, from about 0.004 in to about 0.015 in; and in still other aspects, from about 0.005 in to about 0.012 in. The thickness of base substrate 132 need not be uniform but, rather, may vary along the length of thermal element 130 to achieve a particular configuration of thermal element 130.

Insulating layer 134, which may be an electrically insulating layer, as noted above, may be disposed on either or both sides of base substrate 132. Insulating layer 134 may be a Plasma Electrolytic Oxidation (PEO) coating formed via PEO of either or both sides of base substrate 132. Other suitable materials for insulating layer 134, e.g., PTFE, PEEK, PEI, glass, etc., and/or methods of forming insulating layer 134, e.g., sintering, anodization, deposition, spraying, adhesion, mechanical attachment, etc., on either or both sides of base substrate 132 are also contemplated. Where insulating layer 134 is disposed on both sides of base substrate 132, the sides may be of the same or different materials and/or of the same or different thicknesses. Insulating layer 134 may define a thickness (on either or both sides of base substrate 132), in aspects, from about 0.0005 in to about 0.0015 in; in other aspects, from about 0.0007 in to about 0.0013 in; and in still other aspects, from about 0.0009 in to about 0.0012 in. In aspects wherein an insulating base substrate 132, e.g., ceramic, is utilized, insulating layer 134 may be omitted. Further, in aspects, multiple insulating layers 134 are provided on the same side, e.g., two insulating layers 134 on top of one another, each of which may define a thickness (similar or different from one another) within the above-noted ranges or which may collectively define a thickness within the above-noted ranges. The number of layers 134 and/or thicknesses of layers 134 need not be uniform but, rather, may vary along the length of thermal element 130 to achieve a particular configuration of thermal element 130.

Resistive trace circuit 136, as noted above, is disposed on insulating layer 134 (or directly on base substrate 132 where base substrate 132 itself is electrically insulating) on one side of thermal element 130, although it is also contemplated that resistive trace circuit 136 extend to the other side of thermal element 130 or that a second resistive trace circuit 136 be provided on the other side of thermal element 130. Resistive trace circuit 136 may be formed from, for example, platinum, nichrome, kanthal, combinations thereof, or other suitable metal(s) and is disposed on insulating layer 134 via a deposition process, e.g., sputtering, via screen printing, via sintering, or in any other suitable manner. Conductive heater trace 136 may define a thickness, in aspects, from about 0.1 to 500 microns. The thicknesses (and/or width) of conductive heater trace 136 may vary along the length of thermal element 130 to achieve a particular configuration of thermal element 130. In aspects, a cross-section of thermal element 130 achieves resistance in a range of about 5 ohms to about 100 ohms at room temperature. In aspects, the cross-section achieves an increase in resistance at running temperature, e.g., 550° C., of from about 23 ohms to about 150 ohms with a Temperature Coefficient of Resistance (TCR) ranging from about 1000 ppm/° C. to about 3500 ppm/° C. TCR is defined as a relative change of resistance per degree of temperature change, measured in ppm/° C. (1 ppm=0.0001%), wherein TCR=(R2−R1)/R1 (T2−T1).

Resistive trace circuit 136, as noted above, extends from first end 140a thereof distally along body 131a of thermal element 130, loops back proximally at a distal portion of thermal element 130, and extends proximally along body 131a of thermal element 130 back to second end 140b of resistive trace circuit 136. More specifically, a first portion 137a of resistive trace circuit 136 extends distally from first end 140a to a return portion 137c of resistive trace circuit 136 at the distal portion of thermal element 130, while a second portion 137b of resistive trace circuit 136 extends proximally from return portion 137c to the second end 140b at the proximal portion of thermal element 130. Both the first and second portions 137a, 137b, respectively, extend in substantially longitudinal, linear fashion in generally parallel orientation relative to one another. As detailed below, this configuration, together with the overall construction of thermal element 130, provides a particular thermal gradient profile along the length of thermal element 130 when thermal element 130 is energized.

In aspects, thermal element 130 further includes an encapsulating layer 138 disposed on either or both sides of body 131a of thermal element 130 and/or proximal extension 131b of thermal element 130. For example, encapsulating layer 138 may encapsulate body 131a of thermal element 130 on the side of thermal element 130 including an insulating layer 134 and resistive trace circuit 136, although other configurations are also contemplated. Encapsulating layer 138 may define a thickness (on either or both sides of base substrate 132), in aspects, from about 0.0005 in to about 0.0015 in; in other aspects, from about 0.0007 in to about 0.0013 in; and in still other aspects, from about 0.0009 in to about 0.0012 in. In aspects, the thickness may extend, in any of the ranges above or any other suitable range up to about 0.005 inches. This thickness may be uniform or varied along thermal element 130 to achieve a desired configuration.

In configurations where thermal element 130 is double-sided, e.g., includes, on each side, one or more insulating layers 134, a resistive trace circuit 136, and an encapsulating layer 138, the resistive trace circuits 136 on the first and second sides can be connected through, around, or via the thermal element 130. For example, the insulative layer 134 on the first side may have an opening towards a distal end thereof to expose the base substrate 132, enabling the first resistive trace circuit 136 to make connection thereto. Correspondingly, the insulative layer 134 on the second side may also have an opening towards a distal end thereof to expose the base substrate 132, enabling the second resistive trace circuit 136 to make connection thereto. In such aspects, the base substrate 132 is made at least partially from an electrically-conductive material and thus becomes an electrically-conductive pathway, e.g., a via, between the first and second resistive trace circuits 136. This configuration provides a thermal heater trace loop that starts towards the proximal end of the first side of the thermal element 130, extends distally along the first side, connects through towards the distal end of the second side, and extends proximally along the second side towards the proximal end thereof. Thus, the contacts for connection to the first and second contact clips 139, 141 are provided on opposite sides of the thermal element 130.

Referring still to FIGS. 4 and 5, thermal element 130 may be configured to receive an applied voltage (V_{AC}), e.g., the voltage output from electrosurgical generator “G” (FIG. 1) to thermal element 130, in aspects, from about 5 volts to about 250 volts; in other aspects, from about 10 volts to about 175 volts; and in still other aspects, from about 25 volts to about 100 volts.

Thermal element 130 may be configured to operate in one or more different modes, e.g., controllable/settable at electrosurgical generator “G” (FIG. 1) or on housing 20 (FIG. 1) such as, for example, adjacent to or incorporated with second activation switch 90 (FIG. 1). More specifically, thermal element 130 may have a single operating mode and corresponding operating temperature for all functions, or may have multiple operating modes each having a corresponding operating temperature for one or more functions such as, for example: back scoring, tenting, plunger cutting, jaws open cutting, jaws closed cutting, slow cutting, fast cutting, spot coagulation, etc. The operating temperatures for the one or more operating modes may be similar or different and any or all may be, in aspects, of at least about 350° C.; in other aspects, from about 350° C. to about 550° C.; in yet other aspects, about or at least 550° C.; in still yet other aspects, from about 400° C. to about 500° C.; and in other aspects, from about 425° C. to about 475° C. As detailed below, the temperature of thermal element 130 may vary along the length thereof (thus defining a thermal gradient profile of thermal element 130) in use and, thus, the temperatures noted herein may be average temperatures (e.g., obtained via averaging temperature measurements at several locations along the length of thermal element 130), or may represent the temperature at a particular reference point along thermal element 130 (e.g., at a midpoint).

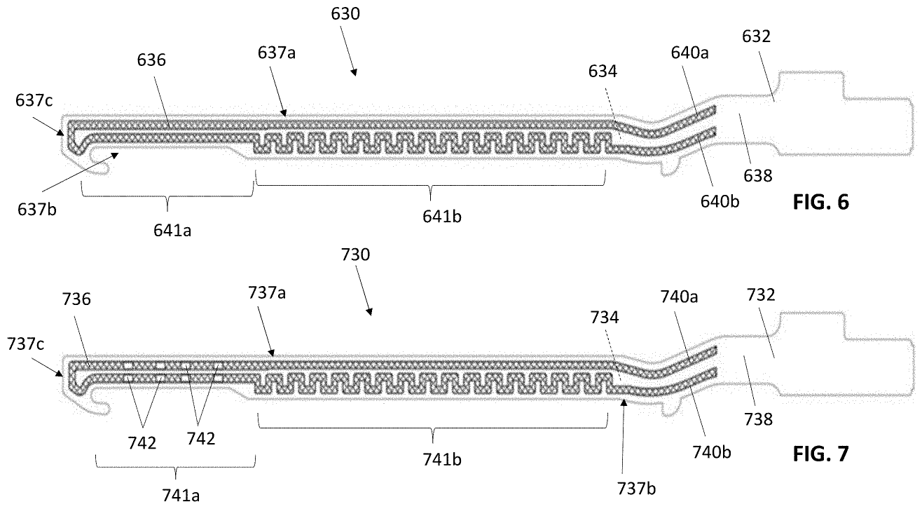

Turning to FIGS. 6-10, various thermal elements 630-1030 (FIGS. 6-10, respectively) provided in accordance with the present disclosure are shown. Thermal elements 630-1030 may be incorporated into an electrosurgical forceps (shaft-based, hemostat-style, or robotic) similarly as detailed above with respect to thermal element 130 (FIGS. 4 and 5), e.g., to function as a thermal cutting element to cut sealed tissue, cut grasped tissue, dissect tissue in an open-jaw configuration, or perform otomies using an exposed distal tip thereof. Alternatively, thermal elements 630-1030 may be configured for use in other surgical instruments and/or for other purposes. For example, one or more similar or different thermal elements 630-1030 may be utilized on one or both jaw members of a forceps-style device to enable thermal tissue sealing and, in aspects, thermal cutting of sealed tissue. As another example, one or more thermal elements 630-1030 may be incorporated into a probe-style device to perform spot coagulation, back scoring, otomies, tissue dissection, etc., similar to the manner in which a monopolar or ultrasonic probe is utilized. Thus, thermal elements 630-1030 may be incorporated into a wide range surgical instruments to facilitate thermally treating tissue in a variety of different ways.

Continuing with reference to FIGS. 6-10, each thermal element 630-1030 may be similar to thermal element 130 (FIG. 5) and include any of the features of thermal element 130 (FIG. 5) except as explicitly contradicted below. Accordingly, only differences between thermal elements 630-1030 and thermal element 130 (FIG. 5) are described in detail below while similarities are summarily described or omitted entirely.

Each thermal element 630-1030 is formed from a base substrate 632-1032 that includes an insulating layer 634-1034 disposed on at least one side of the base substrate 632-1032, and a resistive trace circuit 636-1036 disposed on the insulating layer 634-1034 on the at least one side of the base substrate 632-1032. The resistive trace circuit 636-1036 of each thermal element 630-1030 extends distally along the respective thermal element 630-1030 and loops back proximally such that the first and second ends 640a-1040a, 640b-1040b, respectively, of each of the resistive trace circuits 636-1036 are both disposed at the proximal end portion of the respective thermal element 630-1030, although both ends may alternatively be disposed at the distal end portion of the respective thermal element 630-1030 or otherwise positioned (e.g., without looping the resistive trace circuits 636-1036). Each thermal element 630-1030 may further include an encapsulating layer 638-1038 encapsulating the corresponding insulating layer 634-1034 and resistive trace circuit 636-1036.

The resistive trace circuit 636-1036 of each thermal element 630-1030, more specifically, includes a first portion 637a-1037a extending distally from the respective first end 640a-1040a thereof, a second portion 637b-1037b extending proximally to the respective second end 640b-1040b thereof, and a return portion 637c-1037c interconnecting the first portion 637a-1037a and the second portion 637b-1037b at the distal end portion of the corresponding thermal element 630-1030 such that each resistive trace circuit 636-1036 extends continuously from the first end 640a-1040a thereof at a proximal end portion of the thermal element 630-1030, substantially along the length of the thermal element 630-1030, to the distal end portion of the thermal element 630-1030, and back to the second end 640b-1040b thereof at the proximal end portion of the thermal element 630-1030. As noted above, and although not shown in FIGS. 6-10), first ends 640a-1040a and second ends 640b-1040b connect to or include electrically-conductive layers, traces, pads, etc. to facilitate connection to lead wires for supplying power to thermal element 630-1030, e.g., via welded leads, mechanical connectors, etc. The electrically-conductive traces (or layers, pads, etc.), wherever incorporated into, routed and/or overlaid on the resistive traces to make the electrical connection, are low resistance and thus will not generate resistive heating.

A thermal gradient profile along or otherwise across a thermal element, e.g., thermal element 130 (FIG. 5) or any one of thermal elements 630-1030 (FIGS. 6-10, respectively), may vary in temperature due to the configuration of the thermal element, surrounding structure(s), and/or other factors. Further, depending upon the instrument the thermal element is incorporated into and/or the tissue treatment(s) to be performed with the thermal element, it may be desirable to provide a particular thermal gradient profile (and/or particular temperatures or temperature ranges) along the length of the thermal element (and/or across a width thereof, radially inwardly/outwardly, etc.). Thermal elements 630-1030, as detailed below, are configured to provide different thermal gradient profiles along the lengths thereof and may be utilized to achieve more uniform heating across the thermal elements 630-1030 or to achieve particular variable thermal gradient profiles across the thermal elements 630-1030, depending upon a particular purpose. More uniform heating may be more advantageous, for example, where a substantial entirety of the thermal elements 630-1030 is utilized to treat tissue and/or for treating tissue without first cutting, e.g., for coagulation or sealing. A different thermal gradient profile, on the other hand, may be more advantageous, for example, where only portions of the thermal elements 630-1030 are utilized to treat tissue or where more tissue will typically be concentrated at certain portions of the thermal elements 630-1030 as compared to other portions of the thermal elements 630-1030. However, it is understood that thermal elements 630-1030, the features that facilitate achieving the particular thermal gradient profiles thereof, the thermal gradient profiles provided thereby, and the purposes therefor, are merely exemplary; indeed, it is contemplated that any suitable thermal element 630-1030 may be provided and configured to achieve any suitable thermal gradient profile across the thermal element 630-1030 (whether along the length, width, radially, combinations thereof, or in any other suitable manner) for any suitable purpose.

Referring to FIG. 6, first portion 637a of resistive trace circuit 636 of thermal element 630 extends in substantially longitudinal, linear fashion from along thermal element 630 from first end 640a of resistive trace circuit 636 to return portion 637c of resistive trace circuit 636. Return portion 637c defines a 180 degree turn such that second portion 637b of resistive trace circuit 636 extends proximally back to second end 640b of resistive trace circuit 636. Second portion 637b of resistive trace circuit 636 includes a distal section 641a and a proximal section 641b. Distal section 641a extends in substantially longitudinal, linear fashion along thermal element 630. However, proximal section 641b defines a non-linear, tortuous path such as, for example, a square-wave shaped configuration (as shown), although other suitable configurations are also contemplated such as, for example, a sine-wave shaped configuration, a triangle-wave shaped configuration, a sawtooth-wave shaped configuration, combinations thereof (as mixed wave shapes or discrete wave shapes in a pattern), any of the above shapes with intermediate (periodic or random) longitudinal, linear segments (or other suitable segments), etc. Notably, the tortuous path defined by second portion 637b need not define a waveform-shaped configuration (or combination thereof) and need not define a repeating configuration. As shown in FIG. 6, proximal section 641b defines a square-wave shaped configuration of substantially uniform amplitude and frequency; however, variations are also contemplated. In addition, the linear and tortuous sections of resistive trace circuit 636 detailed above need not be limited to the distal and proximal sections 641a, 641b, respectively; rather, any suitable number and/or arrangement of linear and/or tortuous paths forming first and/or second portions 637a, 637b, respectively, of resistive trace circuit 636 (or other portions thereof) are contemplated, depending upon a particular purpose such as, for example, to achieve a particular thermal gradient profile.

With reference to FIG. 7, resistive trace circuit 736 of thermal element 730 is similar to resistive trace circuit 636 of thermal element 630 (see FIG. 6) and, thus, only differences therebetween are described in detail hereinbelow while similarities are summarily described or omitted entirely.

First portion 737a of resistive trace circuit 736 defines a substantially longitudinal, linear configuration while second portion 737b of resistive trace circuit 736 includes a distal section 741a defining a substantially longitudinal, linear configuration and a proximal section 741b defining a tortuous path. First portion 737a and distal section 741a of second portion 737b of resistive trace circuit 736 each further include a plurality of temperature control features 742 which may be electrically-conductive material disposed on resistive trace circuit 736 and/or areas of increased or decreased thickness of resistive trace circuit 736, encapsulating layer 738, and/or insulating layer 734, etc. With respect to temperature control features 742 configured (at least partially) as electrically-conductive material, such features 742 function to reduce temperature at the locations where provided since the features 742 do not generate resistive heating (because they are conductors) and because current travels through the path of least resistance, thus bypassing the resistive heating portions in favor of the electrically-conductive portions disposed thereon. With respect to temperature control features 742 configured (at least partially) as variations in thickness of resistive trace circuit 736, increased cross-sectional area portions of the resistive trace circuit 736 will have lower resistance and therefore generate less resistive heating at those locations; vice-versa, a decreased cross-sectional area has higher resistance and therefore generates more resistive heating at those locations. Further, although temperature control features 742 are shown disposed only on distal section 741a of second portion 737b of resistive trace circuit 736 and the opposing section of first portion 737a, it is contemplated that any suitable number of temperature control features 742 may be disposed on one or more different portions of resistive trace circuit 736 in any suitable pattern or arrangement so as to facilitate defining a particular thermal gradient profile of resistive trace circuit 736.

Turning to FIG. 8, resistive trace circuit 836 of thermal element 830 is similar to resistive trace circuit 636 of thermal element 630 (see FIG. 6) and, thus, only differences therebetween are described in detail hereinbelow while similarities are summarily described or omitted entirely.

First portion 837a of resistive trace circuit 836 defines a substantially longitudinal, linear configuration while second portion 837b of resistive trace circuit 836 includes a distal section 841a defining a substantially longitudinal, linear configuration and a proximal section 841b defining a tortuous path. Proximal section 841b defines a square-wave shaped configuration, although other suitable configurations are also contemplated; however, the square-wave of proximal section 841b is not uniform but, rather, defines a varied frequency along its length. More specifically, the frequency of the square-wave shape of resistive trace circuit 836 in proximal section 841b of second portion 837b of resistive trace circuit 836 increases in a distal-to-proximal direction at a first rate of increase. As an alternative to increasing distal-to-proximal direction, the frequency may increase in the opposite direction; the frequency may increase to a maximum at a center position (or other position along the length) before decreasing back towards or two the initial frequency; or the frequency may decrease to a minimum at a center position (or other position along the length) before increasing back towards or two the initial frequency.

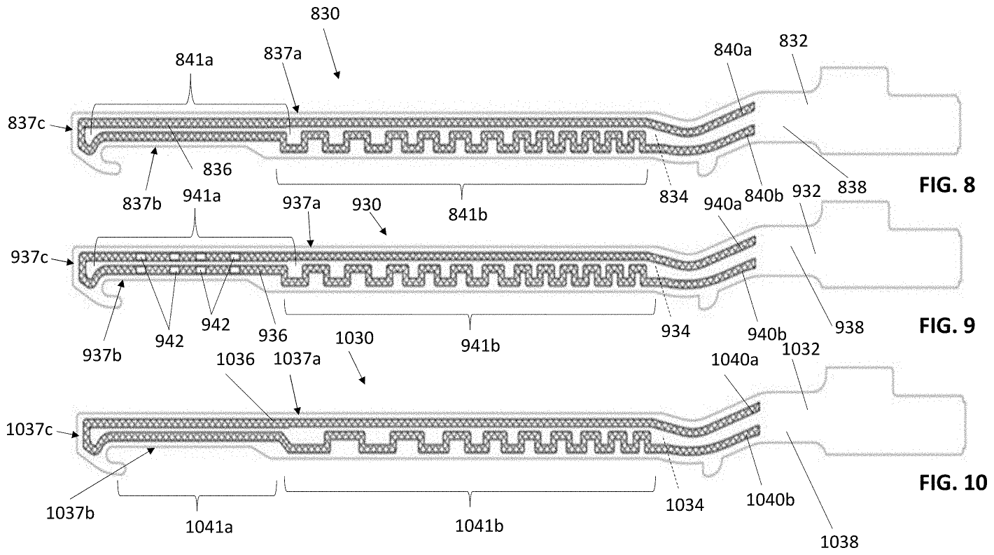

FIG. 9 illustrates another thermal element 930 including a resistive trace circuit 936. Resistive trace circuit 936 is a combination of resistive trace circuit 736 (FIG. 7) and resistive trace circuit 836 (FIG. 8). More specifically, first portion 937a of resistive trace circuit 936 and distal section 941a of second portion 937b of resistive trace circuit 936 include the features, e.g., temperature control features 942, of first portion 737a of resistive trace circuit 736 and distal section 741a of second portion 737b of resistive trace circuit 736 (see FIG. 7), respectively, while proximal section 941b of second portion 937b of resistive trace circuit 936 includes the features, e.g., a square-wave shape configuration having an increasing frequency in a distal-to-proximal direction at the first rate of increase, of proximal section 841b of second portion 837b of resistive trace circuit 836 (see FIG. 8).

Referring to FIG. 10, resistive trace circuit 1036 of thermal element 1030 is similar to resistive trace circuit 836 of thermal element 830 (see FIG. 8) and, thus, only differences therebetween are described in detail hereinbelow while similarities are summarily described or omitted entirely.

First portion 1037a of resistive trace circuit 1036 defines a substantially longitudinal, linear configuration while second portion 1037b of resistive trace circuit 1036 includes a distal section 1041a defining a substantially longitudinal, linear configuration and a proximal section 1041b defining a tortuous path. Proximal section 1041b defines a square-wave shaped configuration, although other suitable configurations are also contemplated; however, the square-wave of proximal section 1041b is not uniform but, rather, defines a varied frequency along its length. More specifically, the frequency of the square-wave shape of resistive trace circuit 1036 in proximal section 1041b of second portion 1037b of resistive trace circuit 1036 increases in a distal-to-proximal direction at a second rate of increase that is greater than the first rate of increase associated with resistive trace circuits 836, 936 of thermal elements 830, 930 (see FIGS. 8 and 9, respectively).

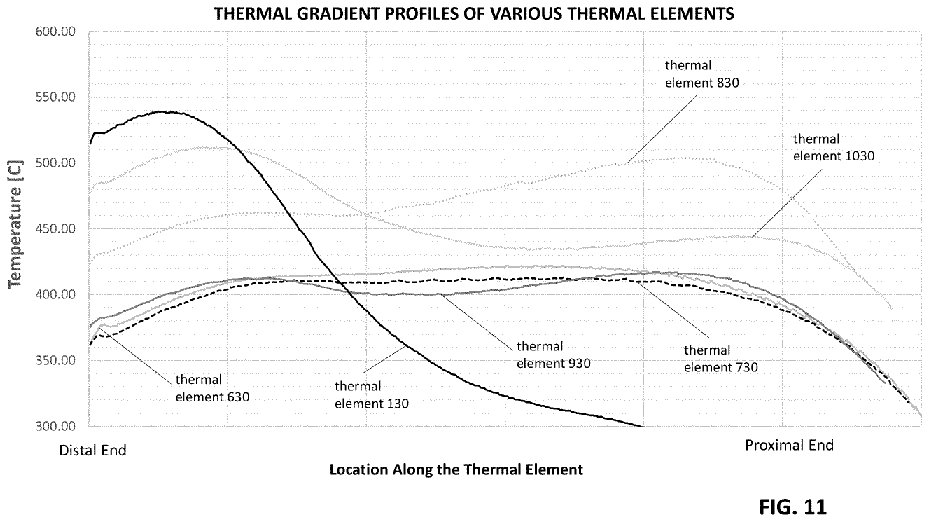

With reference to FIG. 11, thermal gradient profiles of thermal elements 130, 630, 730, 830, 930, and 1030 (FIGS. 5-10) are shown for the same applied energy, e.g., utilizing the same applied voltage, power, or other settings/algorithm. The temperatures of thermal elements 130, 630, 730, 830, 930, and 1030 (FIGS. 5-10) are proportional to the amount of power input from electrosurgical generator (FIG. 1) and, thus, the temperature gradient curves are substantially maintained in shape but will translate vertically on the graph depending upon the power input. As can be appreciated in view of the thermal gradient profiles of FIG. 11 and the configurations of thermal elements 130, 630, 730, 830, 930, and 1030 (FIGS. 5-10) detailed above, the various different features of thermal elements 130, 630, 730, 830, 930, and 1030 and resistive trace circuits 136, 636, 736, 836, 936, 1036 (FIGS. 5-10) thereof provide different thermal gradient profiles (and different temperatures and temperature ranges) along the lengths of thermal elements 130, 630, 730, 830, 930, and 1030 (FIGS. 5-10). Thus, by selecting appropriate features and/or configurations, a thermal element in accordance with the present disclosure can be provided having a desired thermal gradient profile and temperatures or temperature ranges (e.g., for uniform heating along the thermal element, increased heating at one or more locations along the thermal element, tapered heating along the thermal element or portion(s) thereof, etc.).

Continuing with reference to FIG. 11, the thermal gradient profiles of thermal elements 630, 730, and 930, for example, provide a substantially uniform temperature in that the temperature is maintained within 25° C. along at least a majority of the length of each of these thermal elements 630, 730, and 930, wherein the 25° C. range itself is within a range of about 400° C. to about 450° C.

The thermal gradient profile of thermal element 130 defines a peaked shaped having a variation of greater than 100° C. and, in aspects, greater than 150° C. at different locations along thermal element 130. The peak may be disposed towards the distal end of thermal element 130, although other suitable locations are also contemplated. The peak temperature may be about or greater than 500° C.

Thermal elements 830 and 1030 provide dual-peaked configurations and/or central valley configurations wherein the temperatures towards the end portions of the thermal elements 830, 1030 are greater than the temperatures towards the midpoints of the thermal elements 830, 1030. Despite these configurations, the variation along at least a majority of the length of each of these thermal elements 830, 1030 may be within about 100° C. or, in aspects, about 75° C. The peak temperatures may be about or greater than 450° C. or, in aspects, 500° C.

With respect to thermal elements 630-1030, in aspects, the temperature at the proximal end portion and at the distal end portion may be substantially similar such as, for example, within about 25° C., in aspects. In other aspects, the temperature at the proximal end portions and at the distal end portions of thermal elements 630-1030, may vary by less than 15° C., in other aspects, less than 10° C., and in still other aspects, less than 5° C. Other configurations are also contemplated.

While several aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular configurations. Those skilled in the art will envision other modifications within the scope and spirit of the claims appended hereto.

What is claimed is:

1. A thermal element configured for thermally treating tissue, the thermal element comprising:
a substrate;
an insulating layer disposed on the substrate; and
a resistive trace circuit disposed on the insulating layer and including first and second ends adapted to connect to a source of energy for energizing the resistive trace circuit, thereby heating the thermal element,
wherein the resistive trace circuit includes at least first and second sections between the first and second ends, the first and second sections defining different configurations.

2. The thermal element according to claim 1, wherein the first section of the resistive trace circuit defines a substantially linear configuration and wherein the second section of the resistive trace circuit defines a tortuous configuration.

3. The thermal element according to claim 2, wherein the tortuous configuration has a repeating pattern.

4. The thermal element according to claim 3, wherein the repeating pattern varies in frequency.

5. The thermal element according to claim 1, wherein the first and second ends of the resistive trace circuit are disposed at a first end portion of the substrate, and wherein the resistive trace circuit extends from the first end thereof at the first end portion of the substrate to a return portion at a second end portion of the substrate and back from the return portion at the second end portion of the substrate to the second end of the resistive trace circuit at the first end portion of the substrate.

6. The thermal element according to claim 5, wherein the first section is disposed between the first end of the resistive trace circuit and the return portion of the resistive trace circuit and wherein the second section is disposed between the return portion of the resistive trace circuit and the second end of the resistive trace circuit.

7. The thermal element according to claim 1, wherein only one of the first or second sections of the resistive trace circuit includes at least one temperature control feature.

8. The thermal element according to claim 7, wherein the at least one temperature control feature includes electrically-conductive material disposed on the resistive trace circuit.

9. The thermal element according to claim 1, wherein the resistive trace circuit further includes a third section between the first and second ends, and wherein the third section defines a different configuration from both the first section and the second section.

10. The thermal element according to claim 9, wherein one of the first, second, or third sections includes a substantially linear configuration, wherein another of the first, second, or third sections includes a tortuous configuration, and wherein still another of the first, second, or third sections includes a at least one temperature control feature.

11. A thermal element configured for thermally treating tissue, the thermal element comprising:
a substrate;
an insulating layer disposed on the substrate; and
a resistive trace circuit disposed on the insulating layer and including first and second ends adapted to connect to a source of energy for energizing the resistive trace circuit, thereby heating the thermal element,
wherein the resistive trace circuit is configured to maintain a temperature variation of no greater than about 25° C. along at least a majority of a length of the thermal element at a target operating temperature thereof.

12. The thermal element according to claim 11, wherein a first section of the resistive trace circuit defines a substantially linear configuration and wherein a second section of the resistive trace circuit defines a tortuous configuration.

13. The thermal element according to claim 11, wherein the first and second ends of the resistive trace circuit are disposed at a first end portion of the substrate, and wherein the resistive trace circuit extends from the first end thereof at the first end portion of the substrate to a return portion at a second end portion of the substrate and back from the return portion at the second end portion of the substrate to the second end of the resistive trace circuit at the first end portion of the substrate.

14. The thermal element according to claim 13, wherein a first section of the resistive trace circuit disposed between the first end of the resistive trace circuit and the return portion of the resistive trace circuit defines a different configuration from a second section of the resistive trace circuit disposed between the return portion of the resistive trace circuit and the second end of the resistive trace circuit.

15. The thermal element according to claim 11, wherein the resistive trace circuit includes at least one temperature control feature.

16. A thermal element configured for thermally treating tissue, the thermal element comprising:
a substrate;
an insulating layer disposed on the substrate; and
a resistive trace circuit disposed on the insulating layer and including first and second ends adapted to connect to a source of energy for energizing the resistive trace circuit, thereby heating the thermal element,
wherein the resistive trace circuit is configured to define a temperature gradient profile that varies at least about 50° C. along a length of the thermal element at a target operating temperature thereof.

17. The thermal element according to claim 16, wherein a first section of the resistive trace circuit defines a substantially linear configuration and wherein a second section of the resistive trace circuit defines a tortuous configuration.

18. The thermal element according to claim 16, wherein the first and second ends of the resistive trace circuit are disposed at a first end portion of the substrate, and wherein the resistive trace circuit extends from the first end thereof at the first end portion of the substrate to a return portion at a second end portion of the substrate and back from the return portion at the second end portion of the substrate to the second end of the resistive trace circuit at the first end portion of the substrate.

19. The thermal element according to claim 16, wherein the temperature gradient profile includes at least one peak disposed between the first and second ends of the conducive circuit trace at the target operating temperature.

20. The thermal element according to claim 16, wherein the temperature gradient profile includes at least two peaks disposed between the first and second ends of the conducive circuit trace at the target operating temperature.
